(19) United States
(12) Patent Application Publication
(10) Pub. No.: US 2009/0023955 A1
(43) Pub. Date: Jan. 22, 2009

(54) Novel process for the preparation of sertraline hydrochloride form II

(75) Inventors: Daqing Che, Brantford (CA); Fan Wang, Hamilton (CA); Shahid Rabbani, Maple (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Apotex Pharmachem Inc.

(21) Appl. No.: 11/976,466

(22) Filed: Oct. 25, 2007

(30) Foreign Application Priority Data
Jul. 20, 2007 (CA) 2,594,198

(51) Int. Cl. C07C 209/00

(52) U.S. Cl. 564/308

(57) ABSTRACT

The present invention relates to novel processes to produce sertraline hydrochloride Form II comprising the steps of forming a solution of sertraline hydrochloride in a polar organic solvent and adding this solution to a less polar organic solvent.

### FIELD OF THE INVENTION

The present invention relates to novel, industrially suitable processes for the preparation of sertraline hydrochloride Form II.

### BACKGROUND OF THE INVENTION

Sertraline hydrochloride, (1S, 4S)-4-(3,4-dichlorophenyl)-1,2,3,4-tetrahydro-N-methyl-1-naphthalenamine hydrochloride, is an antidepressant and anorectic agent used for treatment of depression, obsessive-compulsive disorder and panic disorder, which is marketed by Pfizer as Zoloft®. Sertraline hydrochloride has the following chemical structure:

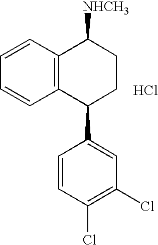

Sertraline hydrochloride was first disclosed in U.S. Pat. No. 4,536,518 (U.S. '518). It was prepared by treating an ethyl acetate and ether solution of sertraline free base with gaseous hydrogen chloride. The resulting solid had a melting range of 243 to 245° C.

U.S. Pat. No. 5,248,699 (U.S. '699) discloses five polymorphs of sertraline hydrochloride, Form I, Form II, Form III, Form IV and Form V, all of which are characterized by single crystal X-ray analysis, powder X-ray diffraction (PXRD), infra-red spectroscopy (IR) and differential scanning calorimetry (DSC). U.S. '699 designates sertraline hydrochloride produced by the method of U.S. '518 as Form II, and discloses that Form II may be prepared by rapid crystallization of sertraline hydrochloride from an organic solvent, such as isopropyl alcohol, ethyl acetate or hexane.

U.S. Pat. No. 6,495,721 (U.S. '721) describes several methods for preparing sertraline hydrochloride Form II. This patent purports that the procedures in U.S. '518 and U.S. '699 do not produce sertraline hydrochloride Form II. The methods disclosed in U.S. '721 are summarized below:
1) Dissolving sertraline free base in an organic solvent; adding hydrogen chloride to the solution; heating the solution to form sertraline hydrochloride Form II; and isolating by filtration.
2) Dissolving sertraline hydrochloride in a solvent selected from N,N-dimethylformamide (DMF), cyclohexanol, acetone, or mixtures thereof; heating the solution to form sertraline hydrochloride Form II; and isolating.
3) Granulating sertraline hydrochloride Form V in ethanol or methanol and stirring to induce formation of sertraline hydrochloride Form II.
4) Suspending a water or solvent adduct of sertraline hydrochloride in a solvent selected from the group consisting of acetone, t-butyl methyl ether (MTBE), cyclohexane, n-butanol and ethyl acetate, to form sertraline hydrochloride Form II, and isolating.
5) Heating sertraline hydrochloride ethanolate Form VI at up to 1 atmosphere pressure and isolating a mixture of Form II and Form V.

These methods have the following deficiencies with respect to commercial scale production:
1) Difficult to control the process to produce pure Form II. For instance, upon prolonged heating or stirring, sertraline hydrochloride Form II will convert to the purportedly more stable Form I.
2) Additional steps required to make other polymorphic forms before producing sertraline hydrochloride Form II.
3) Produces a mixture of polymorphs, which is undesirable in the pharmaceutical industry and may cause difficulties during formulation.

U.S. Pat. No. 6,500,987 (U.S. '987) discloses methods to make sertraline hydrochloride Form II by conversion of other polymorphic forms, such as Form V and Form VI, in an organic solvent. U.S. Pat. No. 6,452,054 (U.S. '054) also discloses methods to make sertraline hydrochloride Form II by conversion of other polymorphic forms, in particular Form XIV, Form XV and Form XVI, in an organic solvent. The processes disclosed by U.S. '987 and U.S. '054 are deficient since again it is necessary to initially obtain other polymorphic forms of sertraline hydrochloride prior to producing sertraline hydrochloride Form II.

U.S. Pat. No. 6,897,340 (U.S. '340) provides a process to make sertraline hydrochloride Form II containing less than 1% Form I by contacting sertraline free base solution with HCl gas at temperatures between 30 and 60° C. It is disclosed in the experimental section of U.S. '340 (examples 7 and 8) that at 70° C. or with filtration at 10° C., the same procedure produces a mixture of sertraline hydrochloride Form I and Form II. Furthermore, this process has a strict requirement that the solid needs to be filtered immediately after adding HCl gas, which is not preferable for commercial scale. Finally, prolonged handling time (which would occur on scale-up) may result in the partial or complete formation of Form I.

U.S. Pat. No. 7,067,700 (U.S. '700) discloses a process to prepare sertraline hydrochloride Form II by adding HCl in ethyl acetate solution or gaseous HCl to a solution of sertraline free base in isopropyl alcohol at about 40 to 60° C. In the preferred embodiment disclosed by U.S. '700, seed crystals are required to control the polymorph formation.

U.S. Pat. No. 6,872,853 (U.S. '853) describes a similar process as U.S. '700, wherein a solution of sertraline free base is seeded with crystals of Form II and a solution of hydrogen chloride is added. U.S. '853 also discloses processes for forming sertraline hydrochloride Form II from a stirred suspension of sertraline hydrochloride Form V, Form CSC1, Form CSC2 or Form T1 with some seeding crystals of Form II, and by drying sertraline hydrochloride alcohol solvate at 0 to 30° C. in high vacuum.

U.S. Pat. No. 7,173,153 (U.S. '153) discloses a process for forming sertraline hydrochloride Form II wherein sertraline, as a base or citrate salt, is dissolved in an organic solvent; HCl, as a gas, aqueous solution or solution in an organic solvent, is added; and sertraline hydrochloride Form II is isolated by filtration.

U.S. Pat. No. 7,189,876 (U.S. '876) discloses a process for forming sertraline hydrochloride Form II from sertraline free base and various amine hydrochlorides in an organic solution. This process suffers from the deficiency that it will produce an amine by-product which must be removed, thereby increasing processing costs.

Despite the fact that numerous processes are known in the prior art, there is still a need to develop a robust and commercially suitable process to produce sertraline hydrochloride Form II.

### SUMMARY OF THE INVENTION

Sertraline hydrochloride is more soluble in a polar organic solvent, such as alcohol, than in non-polar solvents, such as heptanes, MTBE or ethyl acetate. Sertraline hydrochloride Form II is polymorphically less stable than Form I and converts more easily to Form I if stirred in a polar organic solvent, such as alcohol, relative to a non-polar organic solvent, such as heptanes, MTBE or ethyl acetate.

Surprisingly and unexpectedly, it has been found that sertraline hydrochloride Form II can be formed by a reverse addition process, in which a hot solution of sertraline hydrochloride in a polar organic solvent (or mixture of polar organic solvents) is added to a less polar organic solvent (or mixture of less polar organic solvents) at a controlled temperature. The precipitated sertraline hydrochloride Form II can be isolated by filtration. The prior art does not describe such a process for forming sertraline hydrochloride Form II wherein a second, less polar organic solvent is used as an antisolvent.

The present invention relates to novel processes to produce sertraline hydrochloride Form II comprising the steps of forming a solution of sertraline hydrochloride in a polar organic solvent and adding this solution to a less polar organic solvent. If previously heated, the mixture can be cooled. Sertraline hydrochloride Form II is then isolated by filtration.

Preferably, the present invention relates to novel processes to produce sertraline hydrochloride Form II comprising the steps of dissolving sertraline hydrochloride in n-butanol or DMF, adding the solution to MTBE, heptanes, ethyl acetate, methyl isobutyl ketone (MIBK), acetone, or mixtures thereof, and isolating the sertraline hydrochloride Form II by filtration.

### DETAILED DESCRIPTION OF THE INVENTION

The present invention provides novel processes for making sertraline hydrochloride Form II from sertraline hydrochloride. Sertraline hydrochloride may be made from sertraline free base in an organic solvent, with or without isolation. Sertraline free base may be prepared by the methods known in the art, including the methods described in U.S. '518. For instance, sertraline free base can be obtained from sertraline mandelate salt using a method known in the art, such as in U.S. '518, using an organic solvent such as n-butanol. Sertraline hydrochloride can be made by adding 1 equivalent or more of hydrogen chloride gas or hydrogen chloride solution in alcoholic solvents, such as isopropyl alcohol or n-butanol, at high temperature if required.

The sertraline hydrochloride alcohol solution can be clarified and then added to another organic solvent (or mixture of solvents) which is less polar, such as ethyl acetate, MTBE, MIBK, acetone and heptanes, at a controlled temperature, to form sertraline hydrochloride Form II.

The solvent used to form the sertraline hydrochloride solution is an organic solvent in which sertraline hydrochloride is soluble. The preferred solvents are n-butanol or DMF. The volume of organic solvent can be from 1 to 10 volumes, preferably 3 to 6 volumes, per weight of sertraline hydrochloride.

The mixture of sertraline hydrochloride and organic solvent can be heated such that the sertraline hydrochloride completely dissolves and the temperature range can be from room temperature to reflux temperature of the solvent. For n-butanol, the preferred temperature is 80 to 115° C.

The solvent to which the sertraline hydrochloride solution is added can be any organic solvent that is less polar than the first organic solvent, preferably commonly used organic solvents such as C₃ to C₈ esters, C₄ to C₈ ethers, C₃ to C₈ ketones, C₅ to C₁₀ hydrocarbons, C₃ to C₆ alcohols, or mixtures thereof. Examples of solvents include ethyl acetate, MTBE, MIBK, heptanes, acetone, or mixtures thereof. During the addition process, the temperature range can be from room temperature to reflux temperature of this second organic solvent, depending on the formation of sertraline hydrochloride Form II solid. It is found that if the temperature is controlled, sertraline hydrochloride Form II can be formed immediately, instead of being converted to the gel-like material which has been described in the prior art. The preferred temperature is from 20 to 80° C., more preferably the temperature range is from 35 to 75° C., most preferably the temperature range is from 45 to 65° C. The volume of the second organic solvent can be from 2 to 10 volumes, more preferably 2 to 5 volumes, per weight of sertraline chloride.

Upon completion of the addition, the mixture is cooled to a lower temperature, preferably 0 to 20° C., and filtered to isolate sertraline hydrochloride Form II.

In another aspect of the present invention, a novel process for making sertraline hydrochloride Form II from sertraline mandelate salt is provided. Sertraline mandelate salt can be free-based using methods known in the prior art and the sertraline free base can be isolated after removing the organic solvent. Thus, the free base can be dissolved in a polar organic solvent, such as n-butanol or DMF, to form a solution. To this solution, hydrogen chloride in the form of a gas or a solution, including aqueous solution, can be added to form a sertraline hydrochloride solution, at elevated temperature if required. This solution is then added to a second, less polar organic solvent, including but not limited to, ethyl acetate, MTBE, MIBK, heptanes and acetone (or mixtures thereof), to form sertraline hydrochloride Form II.

The present invention is further illustrated in the following examples. However, the present invention should not be construed as limited thereby. One of ordinary skill in the art will understand how to vary the exemplified preparations to obtain the desired results.

### Preparation of Sertraline Hydrochloride Form II Using Ethyl Acetate as Antisolvent

(1) A suspension of sertraline D-mandelate (10 g) in 40 mL n-butanol and 20 ml 5% aqueous sodium hydroxide was stirred at room temperature for 1 hour and the layers were separated. The organic layer was dried over anhydrous Na₂SO₄ and filtered. The filtrate was heated to 110° C. followed by addition of 30% HCl in n-butanol (1.9 g). The hot solution was poured into ethyl acetate (20 mL) and the resulting mixture was maintained at 65 to 70° C. for 30 min. After cooling to room temperature, the sertraline hydrochloride was collected by filtration and dried (6.4 g). Powder X-ray diffraction testing shows the product to be sertraline hydrochloride polymorphic Form II.

(2) A suspension of sertraline hydrochloride (5 g) in 25 mL n-butanol was heated to 114° C. to obtain a solution. The hot solution was poured into 30 mL ethyl acetate (pre-heated to 50° C.). The resulting mixture was maintained between 55° C. and 60° C. for 15 min. After cooling to room temperature, the sertraline hydrochloride as Form II was collected by filtration and dried (4.1 g).

(3) A suspension of sertraline hydrochloride (5 g) in 10 mL DMF was heated to 100° C. to obtain a solution. The hot solution was poured into 40 mL ethyl acetate (pre-heated to 60° C.). The resulting mixture was maintained between 65° C. and 70° C. for 30 min. After cooling to room temperature, the sertraline hydrochloride as Form II was collected by filtration and dried (4.6 g).

### Preparation of Sertraline Hydrochloride Form II Using MTBE as Antisolvent

(1) A suspension of sertraline hydrochloride (5 g) in 25 mL n-butanol was heated to 114° C. to obtain a solution. The hot solution was poured into 25 mL MTBE (pre-heated to 45° C.). The resulting mixture was maintained between 50° C. and 60° C. for 15 min. After cooling to room temperature, the sertraline hydrochloride as Form II was collected by filtration and dried (4.6 g).

(2) A suspension of sertraline D-mandelate (10 g) in 40 mL n-butanol and 20 mL 5% aqueous sodium hydroxide was stirred at room temperature for 1 hour. The layers were separated. The organic layer was dried over anhydrous Na₂SO₄ and filtered. The filtrate was heated to 100° C. followed by addition of 32% aqueous HCl (2.7 g). The hot solution was poured into a solution of 60 mL MTBE (pre-heated to 40° C.) and the resulting mixture was maintained at 55 to 60° C. for 30 min. After cooling to room temperature, the sertraline hydrochloride as Form II was collected by filtration and dried (5.9 g).

### Preparation of Sertraline Hydrochloride Form II Using MIBK as Antisolvent

A suspension of sertraline hydrochloride (5 g) in 25 mL n-butanol was heated to obtain a clear solution (˜114° C.). The hot solution was poured into 30 mL MIBK (pre-heated to 50° C.). The resulting mixture was maintained between 50° C. and 55° C. for 15 min. After cooling to room temperature, the sertraline hydrochloride as Form II was collected by filtration and dried (3.5 g).

### Preparation of Sertraline Hydrochloride Form II Using Heptanes as Antisolvent

A suspension of sertraline D-mandelate (20 g) in 80 mL n-butanol and 40 mL 5% aqueous sodium hydroxide was stirred at room temperature for 1 hour. The layers were separated. The organic layer was dried over anhydrous Na₂SO₄ and filtered. The filtrate was heated to 100° C. followed by addition of 20% HCl/isopropyl alcohol (IPA) (8.4 g). The hot solution was poured into 80 mL heptanes (pre-heated to 52° C.) and the resulting mixture was maintained at 60 to 65° C. for 30 min. After cooling to room temperature, the sertraline hydrochloride as Form II was collected by filtration and dried (11.5 g).

As many changes can be made to the invention without departing from the scope of the invention, it is intended that all material contained herein be interpreted as illustrative of the invention and not in a limiting sense.

1. A process to prepare sertraline hydrochloride Form II comprising:
(a) forming a solution of sertraline hydrochloride by dissolving said sertraline hydrochloride in a first organic solvent, wherein said first organic solvent is a polar organic solvent or mixture thereof; and
(b) adding the solution formed in step (a) to a second organic solvent to form sertraline hydrochloride Form II, wherein said second organic solvent is selected from a C₃ to C₈ ester, C₄ to C₈ ether, C₃ to C₈ ketone, C₅ to C₁₀ hydrocarbon, C₃ to C₆ alcohol and a mixture thereof.

2. The process according to claim 1 wherein said first organic solvent is selected from n-butanol and DMF.

3. The process according to claim 1 or 2 wherein said second organic solvent is selected from MTBE, heptanes, ethyl acetate, MIBK, acetone and a mixture thereof.

4. The process according to claim 3, wherein sertraline hydrochloride Form II is isolated and dried.

5. The process according to claim 3 wherein the mixture formed in step (b) is maintained at a temperature of 20 to 80° C.

6. The process according to claim 3 wherein the mixture formed in step (b) is maintained at a temperature of 45 to 65° C.

7. The process according to claim 3 wherein the mixture formed in step (b) is cooled to enhance precipitation.

8. The process according to claim 3 wherein the volume of the second organic solvent in step (b) is from 2 to 10 volumes per weight of sertraline hydrochloride.

9. A process to prepare sertraline hydrochloride Form II comprising:
(a) forming a solution of sertraline free base in a first organic solvent, wherein said first organic solvent is a polar organic solvent or mixture thereof, by the process of either:
i. adding a base to a solution comprising sertraline mandelate and a first organic solvent; or
ii. adding a base to a solution comprising sertraline mandelate and an organic solvent, removing said organic solvent to isolate sertraline free base and dissolving said isolated sertraline free base in a first organic solvent;
(b) adding hydrogen chloride, in the form of a gas or a solution, to the solution formed in step (a) to form a sertraline hydrochloride solution; and
(c) adding the solution formed in step (b) to a second organic solvent to form sertraline hydrochloride Form II, wherein said second organic solvent is selected from a C₃ to C₈ ester, C₄ to C₈ ether, C₃ to C₈ ketone, C₅ to C₁₀ hydrocarbon, C₃ to C₆ alcohol and a mixture thereof.

10. The process according to claim 9 wherein said first organic solvent is selected from n-butanol and DMF.

11. The process according to claim 9 or 10 wherein said second organic solvent is selected from MTBE, heptanes, ethyl acetate, acetone, MIBK and a mixture thereof.

12. The process according to claim 11, wherein sertraline hydrochloride Form II is isolated and dried.

13. The process according to claim 11 wherein the mixture formed in step (c) is maintained at a temperature of 20 to 80° C.

14. The process according to claim 11 wherein the mixture formed in step (c) is maintained at a temperature of 45 to 65° C.

15. The process according to claim 11 wherein the mixture formed in step (c) is cooled to enhance precipitation.

16. The process according to claim 11 wherein the volume of the second organic solvent in step (c) is from 2 to 10 volumes per weight of the sertraline hydrochloride.

17. A process to prepare sertraline hydrochloride Form II comprising:
(a) forming a solution of sertraline hydrochloride in n-butanol;
(b) adding the solution formed in step (a) to ethyl acetate, MTBE, MIBK or heptanes to form sertraline hydrochloride Form II;
(c) isolating sertraline hydrochloride Form II; and
(d) drying said sertraline hydrochloride Form II.

18. A process to prepare sertraline hydrochloride Form II comprising:
(a) forming a solution of sertraline hydrochloride in DMF;
(b) adding the solution formed in step (a) to ethyl acetate to form sertraline hydrochloride Form II;
(c) isolating sertraline hydrochloride Form II; and
(d) drying said sertraline hydrochloride Form II.
